(19) United States
(12) Patent Application Publication
(10) Pub. No.: US 2005/0019426 A1
(43) Pub. Date: Jan. 27, 2005

(54) Use of an aqueous or hydroalcoholic extract from bauhinia for the preparation of a composition

(76) Inventors: Corinna Wirth, Darmstadt (DE); Herwig Buchholz, Frankfurt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/876,632

(22) Filed: Jun. 28, 2004

(30) Foreign Application Priority Data
Jul. 3, 2003 (DE) 103 29 955.6

(51) Int. Cl. A61K 35/78

(52) U.S. Cl. 424/725

(57) ABSTRACT

The present invention relates to a composition comprising an aqueous or hydroalcoholic extract of bauhinia and to the use of an aqueous or hydroalcoholic extract of bauhinia for the preparation of a composition for the care, preservation or improvement of the general state of the skin or hair, for the prophylaxis or prevention of human skin or human hair ageing processes and for the prophylaxis and/or treatment of diseases associated with skin ageing.

The present invention relates to the use of an aqueous or hydroalcoholic extract of bauhinia for the preparation of a composition for the care, preservation or improvement of the general state of the skin or hair, for the prophylaxis or prevention of human skin or human hair ageing processes and for the prophylaxis and/or treatment of diseases associated with skin ageing. In particular, the present invention relates to the use of a bauhinia extract for the preparation of cosmetic compositions for the prophylaxis and/or prevention of ageing processes of the skin and for wound healing.

The human skin is subject to certain ageing processes, some of which are attributable to intrinsic processes (chronoageing) and some of which are attributable to exogenous factors (environmental, for example photoageing). In addition, temporary or even lasting changes to the skin picture can occur, such as acne, greasy or dry skin, keratoses, rosaceae, light-sensitive, inflammatory, erythematous, allergic or autoimmune-reactive reactions, such as dermatosis and photodermatosis.

The exogenous factors include, in particular, sunlight (UV light) or artificial radiation sources having a comparable spectrum, and compounds which can be formed by the radiation, such as undefined reactive photoproducts, which may also be free-radical or ionic. In addition, the exogenous factors also include harmful environmental influences, such as air contamination and cigarette smoke and the reactive compounds present therein, such as ozone, free radicals, for example the hydroxyl free radical, singlet oxygen and other reactive oxygen or nitrogen compounds which interfere with the natural physiology or morphology of the skin.

Cosmetic or dermatological care products having properties which are claimed to counter the processes described or comparable processes or reduce or reverse the harmful consequences thereof are frequently distinguished by the following specific properties—free-radical-scavenging, antioxidative, inflammation-inhibiting or humectant. They prevent or reduce, inter alia, the activity of matrix-degrading enzymes or regulate the new synthesis of collagen, elastin or proteoglycans.

The use of antioxidants or free-radical scavengers in cosmetic compositions is adequately known per se. Thus, the use of the antioxidative vitamin E in sunscreen formulations is usual. Nevertheless, the effect achieved is even here well short of the hoped-for effect.

Vitamin A and vitamin-A derivatives, such as retinoic acid, retinol and retinol esters, act on the differentiation of epithelial cells and are therefore employed for the prophylaxis and treatment of numerous phenomena which impair the skin state, for example use against acne, psoriasis, senile keratosis, skin discoloration and wrinkles has been described (cf., for example, WO 93/19743 and WO 02/02074). However, retinol has the disadvantage of being very unstable and in addition firstly has to be converted into retinolic acid in order to be active. Retinolic acid is furthermore a strong skin irritant.

Three different theories are discussed for the intrinsic processes of skin ageing, generally known as the “biological clock”. One theory is based on the observation that diploid cells, such as, for example, fibroblasts, have only a limited life in cell culture (Hayflick phenomenon: Campisi J. (1998), The role of cellular senescence in skin aging. J. Investig. Dermatol. Symp. Proc. 3:1-5). The consequence is cellular senescence, which results in modified gene expression and degenerative tissue changes.

A further intrinsic mechanism which contributes to skin ageing is accumulation of free radicals in the cells over the life of an individual and cell damage by these free radicals (Biesalski H. K. (2002), Free radical theory of aging, Curr. Opin. Clin. Nutr. Metab. Care, Volume 5).

The glycosylation theory (Maillard-Theorie: Kasper, M. & Funk, R. H. W. (2001), Age-related changes in cells and tissues due to advanced glycation end products (AGEs), Archives of Gerontology and Geriatrics 32: 233-243; Reiser, K. M. (1998), Nonenzymatic glycation of collagen in aging & diabetes, Proc. Soc. Exp. Biol. Med. 218: 23-37) is today generally recognised as a further intrinsic ageing mechanism (glycosylation, glycosidation or glycation of proteins). Non-enzymatic, spontaneous chemical reactions between proteins and sugars result in a modified structure and functioning of tissue proteins (proteins of the extracellular matrix, for example collagen) and in advanced glycation end products (so-called “AGEs”). Cell properties, such as migration, growth, differentiation, proliferation and gene expression and thus ultimately also the tissue structure are influenced by the modified proteins of the extracellular matrix or AGEs via matrix-cell interactions.

The influence of the above-mentioned exogenous and intrinsic factors can result, inter alia, in direct damage to the DNA of the skin cells and to the collagen, elastin or glycosaminoglycan molecules of the extracellular matrix, which are responsible for the strength of skin. In addition, the signal transduction chains, which are terminated by the activation of matrix-degrading enzymes, may be affected. Important representatives of these enzymes are the matrix metalloproteinases (MMPs, for example collagenases, gelatinases and stromelysins), whose activity is additionally regulated by TIMPs (tissue inhibitors of matrix metalloproteinases).

The consequences of the above-mentioned ageing processes are thinning of the skin, weaker interlacing of epidermis and dermis, and a reduction in the number of cells and the supplying blood vessels. This results in the formation of fine lines and wrinkles, the skin becomes leathery, and pigment defects can occur.

The same factors also act on the hair, where damage can likewise occur. The hairs become brittle, less elastic and dull. The surface structure of the hairs is damaged.

There are various conceivable approaches to inhibition of the above-mentioned AGE formation during protein glycosylation. Inhibitors can act as sugar competitors or modify free amino groups of proteins in order to prevent attachment of sugars. An example of such inhibitors is aspirin, which blocks glycosylation by acetylating lysine radicals. Other inhibitors react with aldose or ketose sugars (protein competitors) and thus prevent Maillard reactions with proteins (AGE formation). This group of inhibitors includes compounds containing free amino groups, such as, for example, the amino acid residues lysine and arginine, and compounds such as carnosine and ethanolamine. The most effective known inhibitor, amino-guanidine, possibly engages in the Maillard cascade in two steps (Khalifah, R. G. et al. (1999), Amadorins: novel post-Amadori inhibitors of advanced glycation reactions, Biochem. Biophys. Res. Commun. 257: 251-258). Thus, amino-guanidine reacts with Amadori compounds, but the inhibition is presumably effected by the inclusion of reactive dicarbonyl intermediates arising from oxidation reactions of free sugars or Amadori products. However, aminoguanidine has unfavourable side effects as hydrazine medicament since it extracts carbonyl compounds which are essential to the body, such as, for example, pyridoxal 5′-phosphate (vitamin B6).

Further inhibitors, such as pyridoxamine or thiamine pyrophosphate, are regarded as post-Amadori inhibitors since they are most effective in inhibiting the conversion of Amadori intermediates into AGEs(Khalifah, R. G. et al. (1999), Amadorins: novel post-Amadori inhibitors of advanced glycation reactions, Biochem. Biophys. Res. Commun. 257: 251-258). Since AGE formation is dependent on oxidation reactions, the use of antioxidants, such as vitamin C or E, or the plant cytokine kinetin is a further approach for the prevention of advanced glycosylation (Verbeke P. et al. (2000), Kinetin inhibits protein oxidation and glycoxidation in vitro. Biochem. Biophys. Res. Commun. 276:1265-1270).

Although the pharmaceutical use of aminoguanidine, pyridoxamine and aspirin is suitable, for example, for the treatment of diabetes, active ingredients disclosed hitherto are of only limited suitability for topical applications. Active ingredients for topical applications of cosmetic products should be sufficiently oxidation- and photostable and should be easy to formulate. Compositions which comprise these active ingredients or active-ingredient combinations should furthermore be extremely well tolerated, and should have no toxicity, no side effects and as far as possible a low irritation potential for the skin. In addition, they should as far as possible have a positive influence on water binding in the skin and maintain or increase the skin elasticity and thus promote smoothing of the skin. In addition, they should preferably produce a pleasant skin feeling on application to the skin.

Owing to the constantly increasing demand for active ingredients for the preventative treatment of human skin and human hair against ageing processes and harmful environmental influences, the object of the present invention was to provide novel active ingredients or active-ingredient combinations which effectively prevent glycosylation of proteins and thus ageing processes in the region of the skin and hair and in addition are suitable for topical application.

Surprisingly, it has been found that an aqueous or hydroalcoholic extract of bauhinia exhibits an extremely good protein glycosylation-inhibiting action and can be used for the treatment and prophylaxis of human skin or human hair ageing processes. Compositions comprising the extract according to the invention are extremely well tolerated and have no toxicity, side effects or irritation potential.

The invention therefore relates to the use of an aqueous or hydroalcohoic extract of bauhinia for the preparation of a composition for the care, preservation or improvement of the general state of the skin or hair.

The invention preferably relates to the use of an aqueous or hydroalcoholic extract of bauhinia for the preparation of a composition for the prophylaxis and/or prevention of human skin or human hair ageing processes, in particular for the prophylaxis, prevention and/or reduction of premature skin ageing, of dry skin, skin unevenness, such as wrinkles, fine lines, rough skin or skin with large pores, and/or pigment defects and keratosis actinica.

The invention preferably also relates to the use of an aqueous or hydroalcoholic extract of bauhinia for the preparation of a composition for the prophylaxis and/or treatment of diseases associated with skin ageing.

The invention furthermore preferably relates to the use of an aqueous or hydroalcoholic extract of bauhinia for the preparation of a composition for the prophylaxis and/or prevention of human skin or human hair ageing processes which are associated with glycosylation of proteins.

The invention additionally also relates to the use of an aqueous or hydroalcoholic extract of bauhinia for the preparation of a composition for the prophylaxis and/or treatment of diseases in the region of the human skin or human hair which are associated with glycosylation of proteins.

The invention also relates to the use of an aqueous or hydroalcoholic extract of bauhinia for the preparation of a composition for the prophylaxis and/or prevention of changes in the properties of the cells or extracellular matrix in the region of the skin or hair which are induced by glycosylation of proteins of the extracellular matrix.

The invention also relates to the use of an aqueous or hydroalcoholic extract of bauhinia for the preparation of a composition for improving wound healing.

The invention also relates, in particular, to the use of an aqueous or hydroalcoholic extract of bauhinia for the preparation of a composition for the prophylaxis and/or treatment of skin diseases which are associated with defective keratinisation relating to differentiation and cell proliferation, in particular for the treatment of acne vulgaris, acne comedonica, polymorphic acne, acne rosaceae, nodular acne, acne conglobata, age-related acne, acne occurring as a side effect, such as acne solaris, medicament-related acne or acne professionalis, for the treatment of other defects of keratinisation, in particular ichthyosis, ichthyosiform states, Darrier's disease, keratosis palmoplantaris, leukoplasia, leukoplasiform states, skin and mucosal (buccal) eczema (lichen), for the treatment of other skin diseases which are associated with defective keratinisation and have an inflammatory and/or immunoallergic component, and in particular all forms of psoriasis relating to the skin, mucous membranes and finger- and toenails, and psoriatic rheumatism and skin atopy, such as eczema, or respiratory atopy, or also hypertrophy of the gums.

The invention additionally relates to the use of an aqueous or hydroalcoholic extract of bauhinia for the preparation of a composition for the prophylaxis and/or treatment of all benign or malignant excrescence of the dermis or epidermis, which may be of viral origin, such as verruca vulgaris, verruca plana, epidermodysplasia verruciformis, oral papillomatosis, papillomatosis florida, and of excrescence which may be caused by UV radiation, in particular basal cell epithelioma and spinal cell epithelioma.

In particular, compositions according to the invention are also suitable for the treatment of other skin diseases, such as dermatitis bullosa and diseases affecting the collagen, for the treatment of certain eye diseases, in particular corneal diseases, for overcoming or combating light-induced skin ageing associated with ageing, for reducing pigmentation and keratosis actinica and for the treatment of all diseases associated with normal ageing or light-induced ageing, for the prevention or healing of wounds/scars of atrophy of the epidermis and/or dermis caused by locally or systemically applied corticosteroids and all other types of skin atrophia, for the prevention or treatment of defects in wound healing, for the prevention or elimination of stretch marks caused by pregnancy or for the promotion of wound healing, for combating defects in tallow production, such as hyperseborrhoea in acne or simple seborrhoea, for combating or preventing cancer-like states or pre-carcinogenic states, in particular promyelocytic leukaemia, for the treatment of inflammatory diseases, such as arthritis, for the treatment of all virus-induced diseases of the skin or other areas of the body, for the prevention or treatment of alopecia, for the treatment of skin diseases or diseases of other areas of the body with an immunological component, for the treatment of cardiovascular diseases, such as arteriosclerosis or hypertension, and of non-insulin-dependent diabetes, and for the treatment of skin problems caused by UV radiation.

The glycosylation theory of ageing was put forward for the first time by A. Cerami (Cerami A. (1985), Hypothesis: Glucose as a Mediator of Aging, Journal of the American Geriatric Society, 33(9) pages 626-634) and Monnier (Hayase F. et al. (1989), Aging of proteins: immunological detection of a glucose-derived pyrrole formed during Maillard reaction in vivo, J. Biol. Chem. Mar. 5; 264 (7):3758-64). Collagen and elastin, the two most important structural proteins of human tissue, undergo, even in the physiological state, molecular changes, such as crosslinking and side-chain modifications. Pyridinoline crosslinking is carried out enzymatically by lysyloxidase. This precise enzymatic process is essential for normal development of the extracellular matrix. By contrast, crosslinking and polymerisation formed non-enzymatically are the result of spontaneous reactions between proteins and sugars. Glycosylating agents here, besides glucose and other sugars, are also oxo aldehydes, glyoxal, methyl glyoxal and 3-deoxyglucosone. Besides proteins, it is also possible for nucleotides and lipids to be spontaneously glycosylated. Glucose in aldehyde form reacts with an amino group of a protein with formation of a Schiff base, which rearranges to give a stable ketoamide (fructosamine) (Amadori rearrangements). These early glycosylation products are crosslinked and polymerised via covalent and irreversible bonds (Maillard reactions), giving brown glycosylation end products (AGEs), in the case of proteins of the extracellular matrix to give proteins having modified chemical and biological properties (abnormal structural stability or denaturing of the proteins). The AGE structures, pentosidine and MOLD (methylglyoxal lysine dimer), are crosslinks between lysine and arginine or between two lysine residues. This affects in particular proteins having a long life, such as, for example, collagen, meaning that chemical damage accumulates with increasing age.

The protein glycosylation described has various adverse effects on the properties of collagen. Thus, it has been shown (Verzijl N. et al. (2000 Sep. 1), Age-related accumulation of Maillard reaction products in human articular cartilage collagen Biochem. J.; 350 Pt 2:381-7) that the accumulation of Maillard products results in stiffer and more brittle collagen. In addition, glycosylation influences correct assembly of collagen monomers to form fibres, and in diabetes patients, significant changes with respect to AGEs in the skin collagen have been found (Beisswenger, P. J. et al. (1993) Diabetes Care 16: 689-694). Glycosylation changes not only the properties of the collagen and of the extracellular matrix, but also modifies the matrix-cell interactions. Since the extracellular matrix determines numerous properties of the adjacent cells, including migration, growth, differentiation and gene expression, these may be adversely affected by changes in the matrix components, such as, for example, non-enzymatic glycosylation of collagen.

Thus, it has been shown that cells cultivated on a matrix of glycosylated proteins generally differ from cells cultivated on a normal matrix with respect to their growth, their differentiation, motility, gene expression and response to cytokines. Various receptors for AGEs (RAGE; OST-48, 80K-H, galectin-3, scavenger receptor) have been found which are expressed on a multiplicity of different cells. Modified cell properties are mediated via the AGE receptors. Thus, cell activation and dysfunctions presumably occur, but the body's defence system against AGEs is presumably directed via these receptors, for example macrophages eliminate modified proteins. Ageing processes are probably also based, inter alia, on a loss of this defence mechanism.

The above-mentioned processes, such as glycosylation of proteins (and other molecules), crosslinking and formation of AGEs and thus modifications to the extracellular matrix (impairment of the structure and function of tissue proteins) and matrix-cell interactions, ultimately result in cell, tissue and organ changes as occur in ageing processes or also in a similar manner in pathophysiology as a consequence of diabetes.

Surprisingly, compositions according to the invention are able effectively to prevent protein glycosylation and ageing processes in the region of the skin and hair.

The aqueous or hydroalcoholic extract used for the preparation of a composition according to the invention can be obtained from all known bauhinia species.

The invention therefore relates to the use according to the invention of an aqueous or hydroalcoholic bauhinia extract for the preparation of a composition, characterised in that the bauhinia extract is obtained from a bauhinia species selected from Bauhinia candicans, Bauhinia championii, Bauhinia fortificata (=Bauhinia forficata), Bauhinia manca, Bauhinia purpurea, Bauhinia racemosa, Bauhinia reticulata, Bauhinia tomentosa, Bauhinia variegata, Bauhinia cheilantha, Bauhinia guanensis, Bauhinia refusa, Bauhinia glauca, Bauhinia pauletia, Bauhinia ungulato, Bauhinia macrostachya and Bauhinia splendens. Preference is given to the extract of Bauhinia variegata. Particular preference is given to the extract obtained from Bauhinia fortificata (=Bauhinia forficata).

The invention preferably relates to the use according to the invention of an aqueous or hydroalcoholic extract of bauhinia for the preparation of a composition, characterised in that the composition comprises an aqueous or hydroalcoholic extract of bauhinia in an amount of from 0.001 to 20% by weight, preferably in an amount of from 0.01 to 10% by weight. The composition particularly preferably comprises an aqueous or hydroalcoholic extract of bauhinia in an amount of from 0.1 to 8% by weight. The person skilled in the art has absolutely no difficulties in selecting the amounts correspondingly depending on the intended effect of the composition.

In principle, all external plant parts can be used for obtaining extracts, but the best results are obtained with extracts from leaves, in particular young leaves of bauhinia. The present invention therefore particularly preferably relates to the use of extracts from young leaves.

Our investigations show that the high efficacy of bauhinia extracts arises from a combination of the various components of the extract.

The following constituents or components have been identified in the extracts:
from Bauhinia candicans: rutin, quercetin, quercitrin, isoquercetin (isoquercitrin), campesterol, stigmasterol, cholesterol, stigmast-3,5-dien-7-one, triacontanol, choline, trigonelline, trigonelline acetate, kaempferol-3-rutinoside, kaempferol-3-rutinoside-7-rhamnoside, sitosterol-3-glycoside (external parts), sitosterol-3-O-β-D-xylopyranoside, sitosterol-3-O-α-D-ribunonosofuranoside, 3-O-methyl-D-inositol (D-pinitol) and sitosterol-3-O-D-xyluronofuranoside;
from Bauhinia championii: 5,6,7,5′-tetramethylenedioxy-3′,4′-methylenedioxyflavone and 5,6,7,5,3′,4′,5′-hexamethoxyflavone-5,7,5′-trimethoxy-3′,4′-methylenedioxyflavone;
from Bauhinia fortificata: quercetin, quercetin-3,7-O-α-dirhamnoside, isoquercetin (isoquercitrin), kaempferol-3-rutinoside, rutin, quercitrin, campesterol, stigmasterol, cholesterol, stigmast-3,5-dien-7-one, triacontanol, choline, trigonelline, kaempferol, kaempferol-7-O-α-rhamnoside, kaempferol-3-rutinoside-7-rhamnoside, kaempferol-3,7-dirhamnoside (kaempferitrin), sitosterol-3-glycoside (external parts), 3-O-methyl-D-inositol (D-pinitol), beta-sitosterol, daucosterol, lupeol, saponins, tannins and astragalin;
from Bauhinia manca: p-coumarinic acid, ferulic acid, phytosterols, cinnamic acid, gallic acid, epicatechin 3-gallate, 5,7-hydroxychromone, hydroxypropioguaiacone, obtustyrene, isoliquitigenine 4-methyl ether, liquiritigenine 4′-methyl ether, 2,4′-dihydroxy-4-methoxydihydrochalcone, 4′-hydroxy-7,3′-dimethoxyflavan, 3′,4′-dihydroxy-7-methoxyflavan, syringaresinol, 5,5′-dimethoxylariciresinol, chrysoeriol and luteolin 5,3′-dimethyl ether;
from Bauhinia purpurea: 6′-(stigmast-5-en-7-one-3-O-glucopyranosidyl) hexa-decanoate, 3-hydroxystigmast-5-en-7-one, oleanolic acid, 6,8-dimethylchrysin, chrysin, isoquercetin (isoquercitrin), astragalin, 2,3-dihydroxypropyl oleate, 2,3-dihydroxypropyl linoleate, 2,3-dihydroxypropyl 16-hydroxyhexadecanoate, 6-butyl-3-hydroxyflavanone, (6-(3″-oxobutyl)taxifolin, 5,6-dihydroxy-7-methoxyflavone and 6-O-D-xylopyranose;
from Bauhinia racemosa: kaempferol, quercetin, kaempferol-3-O-rhamnoside, quercitrin, D-O-methylracemosol and pacharin;
from Bauhinia reticulata: quercetin and quercitrin;
from Bauhinia tomentosa: rutin, quercetin and isoquercetin (isoquercitrin);
from Bauhinia variegata: apigenin, apigenin-7-O-glucoside, kaempferol-3-kaempferol-3-rhamnoglycoside, kaempferol-3-glucoside (astragalin), sitosterol, lupeol, naringenin-4′-rhamnoglucoside, naringin 5,7-dimethyl ether 4′-rhamnoglycoside, 5,7-dihydroxyflavonanone-4′-O-L-rhamnopyranosyl-β-D-gluco-pyranoside (naringenin aglycone), from hydrolysis results: quercetin in leaves, petals, seeds, myricetin in seeds, dihydroxyquercetin (taxifolin) in the pericarp, kaempferol in petals, methyl ester of kaempferol in petals, methyl esters of quercetin in leaves, taxifolin, rutin, quercetin, quercitrin and isoquercetin (isoquercitrin);
from Bauhinia guanensis (in the stem bark): beta-sitosterol, stigmasterol, 3-O-glucopyranosylstigmasta-5,22-diene, 3-O-glucopyranosylsitosterol, 4′-hydroxy-7-methoxyflavan and lapachol.

The present results show that extracts according to the invention are extraordinarily effective in inhibiting protein glycosylation. The efficacy is comparable with the pure active ingredients, such as, for example, aminoguanidine, aspirin or carnosine.

It is assumed that the high efficacy of the extracts arises from the combination of at least two active ingredients. At least one of these active ingredients is presumably a flavone or flavonoid, such as apigenin, apigenin-7-O-glucoside, isoquercetin (isoquercitrin), kaempferol-3-rutinoside, kaempferol-3-galactoside, kaempferol-3-rhamnoglycoside, kaempferol-3-glucoside (astragalin), naringenin-4′-rhamnoglucoside, naringin, quercetin, quercetin-3,7-O-α-dirhamnoside, quercitrin, naringin 5,7-dimethyl ether 4′-rhamnoglycoside, rutin, 5,7-dihydroxyflavanone-4′-O-L-rhamnopyranosyl-β-D-glucopyranoside (naringenin aglycone), preferably with at least one active ingredient being selected from quercetin, isoquercetin and rutin.

Further components of bauhinia extracts according to the invention include polypeptides, polysaccharides, steroids and saponins. In a preferred embodiment of the present invention, the extract used comprises at least one of the above-mentioned active ingredients.

bauhinia extracts according to the invention include all extracts from the above-mentioned bauhinia species prepared by extraction methods known to the person skilled in the art. In particular, the invention also relates to bauhinia extracts which have been further purified by methods known to the person skilled in the art. The invention furthermore also relates to fractions of the said extracts as far as isolated individual compounds which have been isolated from the bauhinia species according to the invention themselves or from extracts thereof, as well as synthetic compounds present in natural form in at least one of the above-mentioned bauhinia species.

Irrespective of the analytical results mentioned, it has been found that, in particular, extracts obtained from parts of bauhinia by extraction with the aid of a mixture of polar solvents and subsequent discarding of the solvent show excellent results.

The invention therefore preferably relates to the use according to the invention of a hydroalcoholic extract of bauhinia, characterised in that the bauhinia extract is obtained by
a) extraction of parts of bauhinia with the aid of a mixture of polar solvents and
b) subsequent discarding of the solvent.

The mixture of polar solvents preferably includes water and at least one further solvent selected from solvents which are less polar than water. A preferred choice of such solvents includes methanol, ethanol, 1-propanol, 2-propanol, acetonitrile, acetone and ethyl acetate. A preferred solvent mixture comprises 10-90% (vol.) of water, particularly preferably 30-70% (vol.) of water.

The extraction can be carried out, for example, as follows:

The effective constituents are extracted from dried bauhinia leaves with the aid of a mixture of ethanol and water having an ethanol content of 50% (vol.). The extraction is carried out at a temperature between 20 and 90° C., preferably between 50 and 90° C., particularly preferably at 70° C. After the solvent has been filtered off, the process can be repeated in order to increase the yield. The extract obtained is in the form of a concentrate. The concentrate is then dried, preferably by spray-drying. Suitable conditions for the spray-drying are described in Example 1.

The invention furthermore preferably relates to the use according to the invention of an aqueous extract of bauhinia, characterised in that the bauhinia extract is obtained by aqueous extraction of parts of bauhinia. The extraction here is carried out by the process indicated above, using water instead of the above-mentioned mixture of polar solvents (hydroalcoholic mixture).

The invention also relates to compositions for preserving or improving the general state of the skin or hair, for the prophylaxis and/or prevention of human skin or human hair ageing processes, in particular for the prophylaxis, prevention and/or reduction of premature skin ageing, of dry skin, skin unevenness, such as wrinkles, fine lines, rough skin or large-pored skin and/or pigment defects and keratosis actinica, for the treatment of diseases associated with skin ageing, for the treatment of diseases associated with skin metabolism and for promoting wound healing, comprising an aqueous or hydroalcoholic extract of bauhinia and at least one further skin-care ingredient and at least one carrier which is suitable for topical applications.

The present invention furthermore relates to the use according to the invention of an aqueous or hydroalcoholic extract of bauhinia for the preparation of a composition, characterised in that the composition, besides the bauhinia extract, comprises at least one further skin-care ingredient and at least one carrier which is suitable for topical applications.

All compounds or components which can be used in the compositions are either known or are commercially available or can be synthesised by known processes. The skin-protecting or skin-care active ingredients can in principle be all active ingredients known to the person skilled in the art.

Skin-care ingredients and carriers are described in greater detail below. Skin-care components are, in particular, antioxidants and/or vitamins.

The invention thus also relates to compositions comprising at least one further skin-care ingredient selected from antioxidants and/or vitamins, preferably selected from vitamin C and derivatives thereof, DL-α-tocopherol, tocopherol E acetate, nicotinic acid, pantothenic acid, biotin, flavonoids, glutathione, coenzyme Q10, protein-bound zinc or selenium and α-lipoic acid.

The present invention furthermore also relates to the use according to the invention of an aqueous or hydroalcoholic extract of bauhinia for the preparation of a composition, characterised in that the composition comprises at least one further skin-care ingredient selected from antioxidants and/or vitamins, preferably selected from vitamin C and derivatives thereof, DL-α-tocopherol, tocopherol E acetate, nicotinic acid, pantothenic acid, biotin, flavonoids, glutathione, coenzyme Q10, protein-bound zinc or selenium and α-lipoic acid.

As described above, oxidative stress or damage by free radicals can cause or influence the various ageing processes. The literature describes that AGE formation can also be dependent on oxidation processes. A further advantage of a bauhinia extract according to the invention is therefore its antioxidative action, which is presumably mediated by the flavonoids present. The protective action of compositions according to the invention against oxidative influences or against the action of free radicals can, however, also be further improved if the compositions comprise one or more further antioxidants, the person skilled in the art having absolutely no difficulties in selecting antioxidants which act suitably quickly or with a delayed action.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), furthermore (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Mixtures of antioxidants are likewise suitable for use in the cosmetic preparations according to the invention. Known and commercial mixtures are, for example, mixtures comprising, as active ingredients, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed with the bauhinia extract in compositions of this type in ratios in the range from 1000:1 to 1:1000, preferably in amounts of from 100:1 to 1:100.

The preparations according to the invention may comprise vitamins as further ingredients. The cosmetic preparations according to the invention preferably comprise vitamins and vitamin derivatives selected from vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active ingredient), thiamine (vitamin B₁), nicotinic acid (niacin), pyridoxine, pyridoxal, pyridoxamine, (vitamin B₆), pantothenic acid, biotin, folic acid and cobalamine (vitamin B₁₂), particularly preferably vitamin C and derivatives thereof, DL-α-tocopherol, tocopherol E acetate, nicotinic acid, pantothenic acid and biotin. Vitamins are usually employed here with the bauhinia extract in ratios in the range from 1000:1 to 1:1000, preferably in amounts of from 100:1 to 1:100.

Of the phenols having an antioxidative action, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′,4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F. Soffers, I. M. C. M. Rietjens; Free Radical Biology&Medicine 2001, 31(7), 869-881, have investigated the pH dependence of the antioxidant action of hydroxyflavones. Quercetin exhibits the greatest activity amongst the structures investigated over the entire pH range.

The invention additionally relates to compositions, characterised in that at least one further skin-care ingredient is a pyrimidinecarboxylic acid and/or aryl oxime, preferably ectoin.

The present invention furthermore relates to the use according to the invention of an aqueous or hydroalcoholic extract of bauhinia for the preparation of a composition, characterised in that at least one further skin-care ingredient is a pyrimidinecarboxylic acid and/or aryl oxime, preferably ectoin.

Pyrimidinecarboxylic acids occur in halophilic microorganisms and play a role in osmoregulation of these organisms (E. A. Galinski et al., Eur. J. Biochem., 149 (1985) pages 135-139). Of the pyrimidinecarboxylic acids, particular mention should be made here of ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoine and ectoine derivatives, such as hydroxyectoin, can advantageously be used in medicaments. In particular, hydroxyectoine can be employed for the preparation of a medicament for the treatment of skin diseases. Other areas of application of hydroxyectoine and other ectoine derivatives are typically in areas in which, for example, trehalose is used as additive. Thus, ectoine derivatives, such as hydroxyectoin, can be used as protectant in dried yeast and bacteria cells. Pharmaceutical products, such as non-glycosylated, pharmaceutically active peptides and proteins, for example t-PA, can also be protected with ectoine or its derivatives.

Of the cosmetic applications, particular mention should be made of the use of ectoine and ectoine derivatives for the care of aged, dry or irritated skin. Thus, European Patent Application EP-A-0 671 161 describes, in particular, that ectoine and hydroxyectoine are employed in cosmetic compositions, such as powders, soaps, surfactant-containing cleansing products, lipsticks, rouge, make-ups, care creams and sunscreen preparations.

Preference is given here to the use of a pyrimidinecarboxylic acid of the following formula II 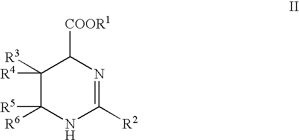
in which R¹ is a radical H or C1-8-alkyl, R² is a radical H or C1-4-alkyl, and R³, R⁴, R⁵ and R₆ are each, independently of one another, a radical from the group consisting of H, OH, NH₂ and C1-4-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid). The compositions according to the invention preferably comprise pyrimidinecarboxylic acids of this type in amounts of up to 15% by weight. The pyrimidinecarboxylic acids are preferably employed here in ratios of from 100:1 to 1:100 with respect to the bauhinia extract, with ratios in the range from 1:10 to 10:1 being particularly preferred.

Of the aryl oximes, preference is given to the use of 2-hydroxy-5-methyllaurophenone oxime, which is also known as HMLO, LPO or F5. Its suitability for use in cosmetic compositions is disclosed, for example, in DE-A-41 16 123. Compositions which comprise 2-hydroxy-5-methyllaurophenone oxime are accordingly suitable for the treatment of skin diseases which are accompanied by inflammation. It is known that compositions of this type can be used, for example, for the therapy of psoriasis, various forms of eczema, irritative and toxic dermatitis, UV dermatitis and further allergic and/or inflammatory diseases of the skin and integumentary appendages. Compositions according to the invention which, in addition to the bauhinia extract, additionally comprise an aryl oxime, preferably 2-hydroxy-5-methyllaurophenone oxime, exhibit surprising antiinflammatory suitability. The compositions here preferably comprise from 0.01 to 10% by weight of the aryl oxime, it being particularly preferred for the composition to comprise from 0.05 to 5% by weight of aryl oxime.

The present invention furthermore relates to compositions comprising at least one further active ingredient selected from tiliroside, aspirin, lysine, arginine, carnosine, ethanolamine, aminoguanidine, pyridoxamine, thiamine pyrophosphate, kinetin, vitamin C and vitamin E.

The present invention also relates to the use according to the invention of an aqueous or hydroalcoholic extract of bauhinia for the preparation of a composition, characterised in that the composition comprises at least one further active ingredient selected from tiliroside, aspirin, lysine, arginine, carnosine, ethanolamine, aminoguanidine, pyridoxamine, thiamine pyrophosphate, kinetin, vitamin C and vitamin E.

Compositions which are particularly preferred in accordance with the invention also comprise UV filters in addition to the bauhinia extract.

The invention therefore relates to compositions comprising one or more UV filters selected from 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate and 2-phenylbenzimidazole-5-sulfonic acid and potassium, sodium and triethanolamine salts thereof, inorganic filters and encapsulated filters.

The present invention also relates to the use according to the invention of an aqueous or hydroalcoholic extract of bauhinia for the preparation of a composition, characterised in that the composition comprises one or more UV filters selected from 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate and 2-phenylbenzimidazole-5-sulfonic acid and potassium, sodium and triethanolamine salts thereof, inorganic filters and encapsulated filters.

In principle, all UV filters are suitable for combination with the bauhinia extract according to the invention. Particular preference is given to UV filters whose physiological acceptability has already been demonstrated. Both for UVA and UVB filters, there are many proven substances which are known from the specialist literature, for example:
benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL),
benzoyl- or dibenzoylmethanes, such as 1-(4-tert-butylphenyl)-3-(4-methoxyphenyl)propane-1,3-dione (for example Eusolex® 9020) or 4-isopropyldibenzoylmethane (for example Eusolex® 8020),
benzophenones, such as 2-hydroxy-4-methoxybenzophenone (for example Eusolex® 4360) or 2-hydroxy-4-methoxybenzophenone-5-sulfonic acid and its sodium salt (for example Uvinul® MS-40),
methoxycinnamic acid esters, such as octyl methoxycinnamate (for example Eusolex® 2292) or isopentyl 4-methoxycinnamate, for example as a mixture of the isomers (for example Neo Heliopan® E 1000),
salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS),
4-aminobenzoic acid and derivatives, such as 4-aminobenzoic acid, 2-ethylhexyl 4-(dimethylamino)benzoate (for example Eusolex® 6007) or ethoxylated ethyl 4-aminobenzoate (for example Uvinul® P25),
phenylbenzimidazolesulfonic acids, such as 2-phenylbenzimidazole-5-sulfonic acid and potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid;
and further substances, such as
2-ethylhexyl 2-cyano-3,3-diphenylacrylate (for example Eusolex® OCR),
3,3′-(1,4-phenylenedimethylene)bis(7,7-dimethyl-2-oxobicyclo[2.2.1]hept-1 -yl-methanesulfonic acid and salts thereof (for example Mexoryl® SX),
2,4,6-trianilino-(p-carbo-2′-ethylhexyl-1′-oxy)-1,3,5-triazine (for example Uvinul® T 150) and
hexyl 2-(4-diethylamino-2-hydroxybenzoyl)benzoate (for example Uvinul® UVA Plus, BASF).

The compounds mentioned in the list should only be regarded as examples. It is of course also possible to use other UV filters.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®),
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),
α-(trimethylsilyl)-ω-[trimethylsilyl)oxy]poly[oxy(dimethyl [and about 6% of methyl[2-[p-[2,2-bis(ethoxycarbonyl]vinyl]phenoxy]-1-methyleneethyl] and approximately 1.5% of methyl[3-[p-[2,2-bis(ethoxycarbonyl)vinyl]phenoxy]propenyl] and from 0.1 to 0.4% of (methylhydrogen]silylene]] (n≈60) (CAS No. 207 574-74-1)
2,2′-methylenebis(6-(2H-benzotriazol-2-yl)-4-(1,1,3,3-tetramethylbutyl)phenol) (CAS No. 103 597-45-1)
2,2′-(1,4-phenylene)bis(1H-benzimidazole-4,6-disulfonic acid, monosodium salt) (CAS No. 180 898-37-7),
2,4-bis{[4-(2-ethylhexyloxy)-2-hydroxy]phenyl}-6-(4-methoxyphenyl)-1,3,5-triazine (CAS No. 103 597-45-, 187 393-00-6) and
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB).

Further suitable UV filters are also methoxyflavones corresponding to the earlier German patent application DE 10232595.2.

Organic UV filters are generally incorporated into cosmetic formulations in an amount of from 0.5 to 20 percent by weight, preferably 1-15%.

Conceivable inorganic UV filters are those from the group consisting of titanium dioxides, such as, for example, coated titanium dioxide (for example Eusolex® T-2000, Eusolex® T-AQUA), zinc oxides (for example Sachtotec®), iron oxides and also cerium oxides. These inorganic UV filters are generally incorporated into cosmetic compositions in an amount of from 0.5 to 20 percent by weight, preferably 2-10%.

Preferred compounds having UV-filtering properties are 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)-benzoate, 2-ethylhexyl 2-cyano-3,3-diphenylacrylate, 2-phenylbenzimidazole-5-sulfonic acid and its potassium, sodium and triethanolamine salts.

Optimised compositions may comprise, for example, the combination of the organic UV filters 4′-methoxy-6-hydroxyflavone with 1-(4-tert-butylphenyl)-3-(4-methoxyphenyl)propane-1,3-dione and 3-(4′-methylbenzylidene)-dl-camphor. This combination gives rise to broad-band protection, which can be supplemented by the addition of inorganic UV filters, such as titanium dioxide microparticles.

All the said UV filters can also be employed in encapsulated form. In particular, it is advantageous to employ organic UV filters in encapsulated form. In detail, the following advantages arise:
The hydrophilicity of the capsule wall can be set independently of the solubility of the UV filter. Thus, for example, it is also possible to incorporate hydrophobic UV filters into purely aqueous compositions. In addition, the oily impression on application of the composition comprising hydrophobic UV filters, which is frequently regarded as unpleasant, is suppressed.
Certain UV filters, in particular dibenzoylmethane derivatives, exhibit only reduced photostability in cosmetic compositions. Encapsulation of these filters or compounds which impair the photostability of these filters, such as, for example, cinnamic acid derivatives, enables the photostability of the entire composition to be increased.
Skin penetration by organic UV filters and the associated potential for irritation on direct application to the human skin is repeatedly being discussed in the literature. The encapsulation of the corresponding substances which is proposed here suppresses this effect.
In general, encapsulation of individual UV filters or other ingredients enables composition problems caused by the interaction of individual composition constituents with one another, such as crystallisation processes, precipitation and agglomerate formation, to be avoided since the interaction is suppressed.

It is therefore preferred in accordance with the invention for one or more of the above-mentioned UV filters to be in encapsulated form. It is advantageous here for the capsules to be so small that they cannot be viewed with the naked eye. In order to achieve the above-mentioned effects, it is furthermore necessary for the capsules to be sufficiently stable and the encapsulated active ingredient (UV filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules which can particularly preferably be employed in accordance with the invention have walls which can be obtained by a sol-gel process, as described in the applications WO 00/09652, WO 00/72806 and WO 00/71084. Preference is again given here to capsules whose walls are built up from silica gel (silica; undefined silicon oxide hydroxide). The production of corresponding capsules is known to the person skilled in the art, for example from the cited patent applications, whose contents expressly also belong to the subject-matter of the present application.

The capsules are preferably present in compositions according to the invention in amounts which ensure that the encapsulated UV filters are present in the composition in the above-indicated amounts.

bauhinia extracts according to the invention can be incorporated into cosmetic or dermatological compositions in the customary manner. Suitable compositions are those for external use, for example in the form of a cream, lotion or gel or as a solution which can be sprayed onto the skin. Suitable for internal use are administration forms such as capsules, coated tablets, powders, tablet solutions or solutions.

Possible use forms of compositions according to the invention are, for example, solutions, suspensions, emulsions, PIT emulsions, pastes, ointments, gels, creams, lotions, powders, soaps, surfactant-containing cleansing preparations, oils, aerosols and sprays. Examples of other use forms are sticks, shampoos and shower compositions. Any desired customary carriers, assistants and, if desired, further active ingredients may be added to the composition.

Preferred assistants originate from the group consisting of preservatives, antioxidants, stabilisers, solubilisers, vitamins, colorants and odour improvers.

Ointments, pastes, creams and gels may comprise the customary carriers, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary carriers, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary carriers, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary carriers, such as liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary carriers, such as alkali metal salts of fatty acids, salts of fatty acid monoesters, fatty acid protein hydrolysates, isethionates, lanolin, fatty alcohol, vegetable oils, plant extracts, glycerol, sugars, or mixtures of these substances.

Surfactant-containing cleansing products may comprise the customary carriers, such as salts of fatty alcohol sulfates, fatty alcohol ether sulfates, sulfosuccinic acid monoesters, fatty acid protein hydrolysates, isethionates, imidazolinium derivatives, methyl taurates, sarcosinates, fatty acid amide ether sulfates, alkyl-amidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary carriers, such as synthetic oils, such as fatty acid esters, fatty alcohols, silicone oils, natural oils, such as vegetable oils and oily plant extracts, paraffin oils or lanolin oils, or mixtures of these substances.

Further typical cosmetic use forms are also lipsticks, lip-care sticks, mascara, eye-liner, eye-shadow, rouge, powder make-up, emulsion make-up and wax make-up, and sunscreen, pre-sun and after-sun preparations.

The preferred composition forms according to the invention include, in particular, emulsions.

Emulsions according to the invention are advantageous and comprise, for example, the said fats, oils, waxes and other fatty substances, as well as water and an emulsifier, as usually used for a composition of this type.

The lipid phase may advantageously be selected from the following group of substances:
mineral oils, mineral waxes;
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group consisting of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, or from the group consisting of esters of aromatic carboxylic acids and saturated and/or unsaturated, branched and/or unbranched alcohols having a chain length of from 3 to 30 carbon atoms. Ester oils of this type can then advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group consisting of branched and unbranched hydrocarbons and waxes, silicone oils, dialkyl ethers, or the group consisting of saturated and unsaturated, branched and unbranched alcohols, and fatty acid triglycerides, specifically the triglycerol esters of saturated and/or unsaturated, branched and/or unbranched alkanecarboxylic acids having a chain length of from 8 to 24, in particular 12-18, carbon atoms. The fatty acid triglycerides may advantageously be selected, for example, from the group consisting of synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The oil phase is advantageously selected from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride and dicapryl ether.

Particularly advantageous are mixtures of C₁₂₋₁₅-alkyl benzoate and 2-ethylhexyl isostearate, mixtures of C₁₂₋₁₅-alkyl benzoate and isotridecyl isononanoate, as well as mixtures of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Furthermore, the oil phase may also advantageously have a content of cyclic or linear silicone oils or consist entirely of oils of this type, although it is preferred to use an additional content of other oil-phase components in addition to the silicone oil or the silicone oils.

The silicone oil to be used in accordance with the invention is advantageously cyclomethicone (octamethylcyclotetrasiloxane). However, it is also advantageous for the purposes of the present invention to use other silicone oils, for example hexamethylcyclotrisiloxane, polydimethylsiloxane or poly(methylphenylsiloxane).

Also particularly advantageous are mixtures of cyclomethicone and isotridecyl isononanoate and of cyclomethicone and 2-ethylhexyl isostearate.

The aqueous phase of the compositions according to the invention optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol or glycerol, and, in particular, one or more thickeners, which may advantageously be selected from the group consisting of silicon dioxide, aluminium silicates, polysaccharides and derivatives thereof, for example hyaluronic acid, xanthan gum, hydroxypropylmethylcellulose, particularly advantageously from the group consisting of the polyacrylates, preferably a polyacrylate from the group consisting of the so-called Carbopols, for example Carbopol grades 980, 981, 1382, 2984 or 5984, in each case individually or in combination.

In particular, mixtures of the above-mentioned solvents are used. In the case of alcoholic solvents, water may be a further constituent.

Emulsions according to the invention are advantageous and comprise, for example, the said fats, oils, waxes and other fatty substances, as well as water and an emulsifier, as usually used for a formulation of this type.

In a preferred embodiment, the compositions according to the invention comprise hydrophilic surfactants.

The hydrophilic surfactants are preferably selected from the group consisting of the alkylglucosides, acyl lactylates, betaines and coconut amphoacetates.

The alkylglucosides are themselves advantageously selected from the group consisting of the alkylglucosides which are distinguished by the structural formula 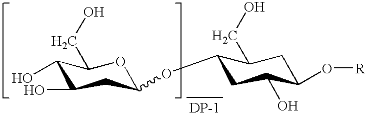
where R is a branched or unbranched alkyl radical having from 4 to 24 carbon atoms, and where \overline{DP} denotes a mean degree of glucosylation of up to 2.

The value \overline{DP} represents the degree of glucosidation of the alkylglucosides used in accordance with the invention and is defined as

$$\overline{DP} = \frac{{p}_{1}}{100} \cdot 1 + \frac{{p}_{2}}{100} \cdot 2 + \frac{{p}_{3}}{100} \cdot 3 + … = \sum \frac{{p}_{i}}{100} \cdot i$$

in which p₁, p₂, p₃ . . . pᵢ represent the proportion of mono-, di-, tri- . . . i-fold glucosylated products in percent by weight. Products which are advantageous according to the invention are those having degrees of glucosylation of 1-2, particularly advantageously of from 1.1 to 1.5, very particularly advantageously of 1.2-1.4, in particular of 1.3.

The value DP takes into account the fact that alkylglucosides are generally, as a consequence of their preparation, in the form of mixtures of mono- and oligoglucosides. A relatively high content of monoglucosides, typically in the order of 40-70% by weight, is advantageous in accordance with the invention.

Alkylglycosides which are particularly advantageously used for the purposes of the invention are selected from the group consisting of octyl glucopyranoside, nonyl glucopyranoside, decyl glucopyranoside, undecyl glucopyranoside, dodecyl glucopyranoside, tetradecyl glucopyranoside and hexadecyl glucopyranoside.

It is likewise advantageous to employ natural or synthetic raw materials and assistants or mixtures which are distinguished by an effective content of the active ingredients used in accordance with the invention, for example Plantaren® 1200 (Henkel KGaA), Oramix® NS 10 (Seppic).

The acyllactylates are themselves advantageously selected from the group consisting of the substances which are distinguished by the structural formula 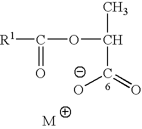
where R¹ is a branched or unbranched alkyl radical having from 1 to 30 carbon atoms, and M⁺ is selected from the group consisting of the alkali metal ions and the group consisting of ammonium ions which are substituted by one or more alkyl and/or one or more hydroxyalkyl radicals, or corresponds to half an equivalent of an alkaline earth metal ion.

For example, sodium isostearyl lactylate, for example the product Pathionic® ISL from the American Ingredients Company, is advantageous.

The betaines are advantageously selected from the group consisting of the substances which are distinguished by the structural formula 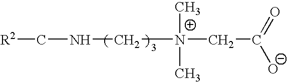
where R² is a branched or unbranched alkyl radical having from 1 to 30 carbon atoms.

R² is particularly advantageously a branched or unbranched alkyl radical having from 6 to 12 carbon atoms.

For example, capramidopropylbetaine, for example the product Tego® Betain 810 from Th. Goldschmidt AG, is advantageous.

A coconut amphoacetate which is advantageous for the purposes of the invention is, for example, sodium coconut amphoacetate, as available under the name Miranol® Ultra C32 from Miranol Chemical Corp.

The compositions according to the invention are advantageously characterised in that the hydrophilic surfactant(s) is (are) present in concentrations of 0.01-20% by weight, preferably 0.05-10% by weight, particularly preferably 0.1-5% by weight, in each case based on the total weight of the composition.

Cosmetic and dermatological compositions according to the invention may exist in various forms. Thus, they may be, for example, a solution, a water-free composition, an emulsion or microemulsion of the water-in-oil (W/O) or oil-in-water (O/W) type, a multiple emulsion, for example of the water-in-oil-in-water (W/O/W) type, a gel, a solid stick, an ointment or an aerosol. It is also advantageous to administer ectoins in encapsulated form, for example in collagen matrices and other conventional encapsulation materials, for example as cellulose encapsulations, in gelatine, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions according to the invention.

Co-emulsifiers which are advantageous according to the invention are, for example, O/W emulsifiers, principally from the group consisting of the substances having HLB values of 11-16, very particularly advantageously having HLB values of 14.5-15.5, so long as the O/W emulsifiers have saturated radicals R and R′. If the O/W emulsifiers have unsaturated radicals R and/or R′ or in the case of isoalkyl derivatives, the preferred HLB value of such emulsifiers may also be lower or higher.

It is advantageous to select the fatty alcohol ethoxylates from the group consisting of ethoxylated stearyl alcohols, cetyl alcohols, cetylstearyl alcohols (cetearyl alcohols). Particular preference is given to the following: polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), poly-ethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, poly-ethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, poly-ethylene glycol (14) oleate, polyethylene glycol (15) oleate, polyethylene glycol (16) oleate, polyethylene glycol (17) oleate, polyethylene glycol (18) oleate, poly-ethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth-14 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group consisting of polyethylene glycol (20) glyceryl laurate, poly-ethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to select the sorbitan esters from the group consisting of polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

Optional W/O emulsifiers, but ones which may nevertheless be advantageous for the purposes of the invention can be the following:
fatty alcohols having from 8 to 30 carbon atoms, monoglycerol esters of saturated and/or unsaturated, branched and/or unbranched alkanecarboxylic acids having a chain length of from 8 to 24 carbon atoms, in particular 12-18 carbon atoms, diglycerol esters of saturated and/or unsaturated, branched and/or unbranched alkanecarboxylic acids having a chain length of from 8 to 24 carbon atoms, in particular 12-18 carbon atoms, monoglycerol ethers of saturated and/or unsaturated, branched and/or unbranched alcohols having a chain length of from 8 to 24 carbon atoms, in particular 12-18 carbon atoms, diglycerol ethers of saturated and/or unsaturated, branched and/or unbranched alcohols having a chain length of from 8 to 24 carbon atoms, in particular 12-18 carbon atoms, propylene glycol esters of saturated and/or unsaturated, branched and/or unbranched alkanecarboxylic acids having a chain length of from 8 to 24 carbon atoms, in particular 12-18 carbon atoms, and sorbitan esters of saturated and/or unsaturated, branched and/or unbranched alkanecarboxylic acids having a chain length of from 8 to 24 carbon atoms, in particular 12-18 carbon atoms.

Particularly advantageous W/O emulsifiers are glyceryl monostearate, glyceryl monoisostearate, glyceryl monomyristate, glyceryl monooleate, diglyceryl mono-stearate, diglyceryl monoisostearate, propylene glycol monostearate, propylene glycol monoisostearate, propylene glycol monocaprylate, propylene glycol mono-laurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate and glyceryl monocaprylate.

Preferred compositions according to the invention are particularly suitable for protecting human skin against ageing processes. In this connection, they are in the various administration forms usually used for this application. For example, they may, in particular, be in the form of a lotion or emulsion, such as in the form of a cream or milk (O/W, W/O, O/W/O, W/O/W), in the form of oily-alcoholic, oily-aqueous or aqueous-alcoholic gels or solutions, in the form of solid sticks or may be formulated as an aerosol.

The composition may comprise cosmetic adjuvants which are usually used in this type of composition, such as, for example, thickeners, softeners, moisturisers, surfactants, emulsifiers, preservatives, antifoams, perfumes, waxes, lanolin, propellants, dyes and/or pigments which colour the composition itself or the skin, and other ingredients usually used in cosmetics.

The dispersant or solubiliser used can be an oil, wax or other fatty substance, a lower monoalcohol or lower polyol or mixtures thereof. Particularly preferred monoalcohols or polyols include ethanol, isopropanol, propylene glycol, glycerol and sorbitol.

A preferred embodiment of the invention is an emulsion in the form of a protective cream or milk which, apart from the bauhinia extract according to the invention, comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The composition according to the invention may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other fatty substances.

In order to enable compositions according to the invention to develop their positive action on the skin particularly well, it may be preferred to allow the bauhinia extract according to the invention to penetrate into deeper skin layers. A number of possibilities are available for this purpose. Firstly, the bauhinia extract may have sufficient lipophilicity in order to be able to penetrate through the outer skin layer into epidermal layers. As a further possibility, corresponding transport means, for example liposomes, can also be provided in the composition, facilitating transport of the active ingredients through the outer skin layers.

In likewise preferred embodiments of the invention, however, the compositions according to the invention may also comprise bauhinia extract which has low solubility or is insoluble in the composition matrix. In this case, the bauhinia extract is preferably dispersed in finely divided form in the cosmetic composition.

If a composition is formulated as an aerosol, the customary propellants, such as alkanes, fluoroalkanes and chlorofluoroalkanes, are generally used.

The cosmetic composition may also be used to protect the hair. In this case, a suitable formulation is in the form of a rinse-out shampoo, lotion, gel or emulsion, the composition in question being applied before or after shampooing, before or after colouring or bleaching or before or after permanent waving. It is also possible to select a composition in the form of a lotion or gel for styling or treating the hair, in the form of a lotion or gel for brushing or blow-waving, in the form of a hair lacquer, permanent waving composition, colorant or bleach for the hair. Besides the bauhinia extract, a composition according to the invention having light-protection properties may comprise various adjuvants used in this type of composition, such as surfactants, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used for hair care.

For use, the cosmetic and dermatological compositions according to the invention are applied in sufficient amount to the skin and/or hair in the usual manner for cosmetics.

The present invention additionally relates to a process for the preparation of a composition, characterised in that an aqueous or hydroalcoholic extract of bauhinia is mixed with a cosmetically or dermatologically suitable carrier. The mixing may result in dissolution, emulsification or dispersion of the bauhinia extract in the carrier.

The compositions according to the invention can be prepared with the aid of techniques which are well known to the person skilled in the art.

The invention is explained in greater detail below with reference to examples. The invention can be carried out throughout the range claimed and is not restricted to the examples mentioned here.

### EXAMPLE 1

### Preparation of a Dry Extract of Bauhinia Forficata with the Aid of Hydroalcoholic Extraction

1. Hydroalcoholic Extraction with 50% Ethanol (v/v)

1 kg of dry, ground leaves of Bauhinia fortificata and 6 l of a 50% hydroalcoholic solution (v/v) are introduced into a glass reaction vessel and refluxed for about one hour with continuous stirring. The extract is filtered using a Büchner porcelain filter with a filter paper.

In a second extraction step, the same volume of 50% hydroalcoholic solution (v/v) is added to the remaining volume in the glass reaction vessel, the mixture is refluxed for about one hour with continuous stirring and filtered using a Büchner porcelain filter with a filter paper.

2. Pre-Concentration

The extract obtained above is concentrated under vacuum (500 mmHg) to a total solids content of between 5.0% and 6.0% in a glass evaporator/concentrator.

3. Spray Drying

The concentrate obtained is spray-dried in a B-191 (Büchi) mini spray drier under the following conditions:
Inlet temperature: 120° C.
Outlet temperature: 65-70° C.
Pump: 15%
Aspirator: 90%
Air inflow: 400 ml/min
Pump flow rate: ˜220 ml/h
Cleaning temporiser: 2
Line pressure: 80 psi (5.5 bar)
   4. Analysis

The extracts are characterised with the aid of high-pressure liquid chromatography (HPLC) and thin-layer chromatography (TLC).

### EXAMPLE 2

### Antioxidative Potential of the Hydroalcoholic Bauhinia Extract

The antioxidative potential of the hydroalcoholic extract from Example 1 is determined as Trolox equivalent antioxidant action (TEAC assay), EC50 (DPPH assay) and relative antioxidant efficiency (RAE; lipid assay) by the method of Halliwell, B.; Eschbach, R.; Löhliger, J.; Aruoma, O. I.; Food. Chem. Toxicol. Vol. 33, pp. 601-67, 1995 (Table 8).

TABLE 1
Antioxidative potential of the extract from Example 1

| Method | Result |
|---|---|
| TEAC assay | TEAC = 0.12 (based on mg/l) |
| DPPH assay | EC50 = 0.37 (based on mg/l to μmol/l) |
| Lipid assay | RAE = 0.19 |

### EXAMPLE 3

### Action of a Hydroalcoholic Bauhinia Extract

In order to stimulate collagen production, human fibroblasts are cultivated with vitamin C. The cells are then lysed by serial freezing/thawing and labelled with D-[5-³H]-glucose over 15 days. At the end of the incubation, the proteins are extracted and precipitated. The pellet is then washed, and at the end of the experiment the glucose covalently bonded to the fibres is measured by liquid scintillation.

The hydroalcoholic bauhinia extract (see Example 1) exhibits a significant reduction in protein glycosylation (55% of the control) at a concentration of 0.03 mg/ml.

### EXAMPLE 4

### Preparation of a Dry Extract of Bauhinia Forficata with the Aid of Aqueous Extraction

A dry extract of Bauhinia forficata is prepared by aqueous extraction by the above-mentioned method for the preparation of a hydroalcoholic extract, but using water as extractant instead of a polar mixture (hydroalcoholic mixture).

### EXAMPLE 5

### Action of an Aqueous Bauhinia Extract

Material:
human dermal fibroblasts (NDHF Pool PF2)
culture medium: DMEM, 2 mM glutamine, 50 Ul/ml of penicillin, 50 μg/ml of streptomycin, 10% foetal calf serum (Life Technologies)
dry extract of Bauhinia forficata according to Example 4 (aqueous extraction), dissolved in sterile PBS
final concentrations tested: 0.3 mg/ml, 0.1 mg/ml, 0.03 mg/ml
reference: aminoguanidine 1 mg/ml

Method:

Human dermal fibroblasts are cultivated to confluence in cell culture medium (see above) in 25 cm² plates at 37° C. and 5% CO₂. Collagen synthesis is stimulated by cultivation of the cells in the presence of vitamin C (final concentration: 20 μg/ml) for 96 hours. The cells are washed with PBS and lysed by serial freezing/thawing. The cells are treated with aqueous extract of Bauhinia forficata or aminoguanidine (and control: PBS) and labelled with 20 μCi/ml D-[5-³H]-glucose (19 Ci/mmol), 703 Gbq/mmol) in the presence of 0.1 mg/ml of glucose. The treatment and labelling are carried out without interruption for 15 days at 37° C. and 5% CO₂. At the end of the incubation, proteins of the extracellular matrix (principally collagen) are extracted by buffer (TRIS 50 mM, 4M guanidine, 5 mM EDTA, pH 8.0, 30 min). The total protein is precipitated by 20% (v/v) trichloroacetic acid, 0.66 mg/ml of albumin. The precipitates are recovered by filtration from a Skatron collector and washed copiously, firstly in 3% trichloroacetic acid, then with 70% ethanol. The glucose covalently bonded to the fibres of the extracellular matrix is finally measured by scintillation.

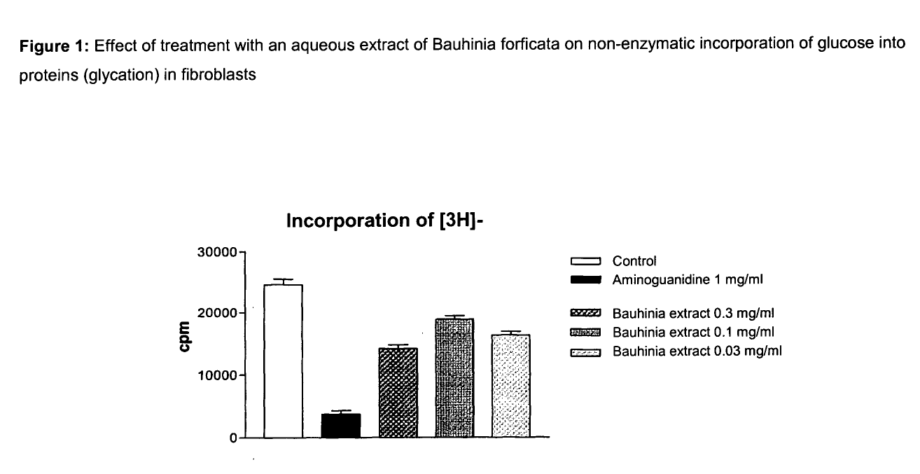

Results and Discussion: (See Also Table 1 and FIG. 1)

Basal non-enzymatic incorporation of glucose into proteins (greater than 20,000 cpm/well) gives an adequate comparison value for investigation of products which influence protein glycation. Aminoguanidine (1 mg/ml) as reference value reduces glucose incorporation by 85%. This value validates the assay. The aqueous extract of Bauhinia forficata (0.3 mg/ml, 0.1 mg/ml and 0.03 mg/ml) also reduces non-enzymatic incorporation of glucose into proteins significantly (58%, 77% and 66% respectively of the control, p<0.01). Consequently, an aqueous extract of Bauhinia forficata in the tested concentrations (0.3 mg/ml, 0.1 mg/ml and 0.03 mg/ml) was capable of reducing glycation of proteins of the extracellular matrix in vitro.

TABLE 2
Effect of the treatment with an aqueous extract of Bauhinia forficata on non-
enzymatic incorporation of glucose into proteins (glycation) in fibroblasts

| | | | Standard | | | |
|---|---|---|---|---|---|---|
| Treatment | cpm | Mean | deviation | n | % of control | p |
| Control | 27267 | 24591 | 2273 | 6 | 100 | — |
| | 23736 | | | | | |
| | 21282 | | | | | |
| | 25147 | | | | | |
| | 23290 | | | | | |
| | 26823 | | | | | |
| Aminoguanidine | 2859 | 3701 | 1002 | 3 | 15 | p < 0.0 |
| 1 mg/ml | 3434 | | | | | |
| | 4809 | | | | | |
| Aqueous extract of | 13101 | 14183 | 996 | 3 | 58 | p < 0.0 |
| Bauhinia forficata | 14384 | | | | | |
| 0.3 mg/ml | 15063 | | | | | |
| Aqueous extract of | 19807 | 18812 | 975 | 3 | 77 | p < 0.0 |
| Bauhinia forficata | 18771 | | | | | |
| 0.1 mg/ml | 17859 | | | | | |
| Aqueous extract of | 17463 | 16256 | 1063 | 3 | 66 | p < 0.0 |
| Bauhinia forficata | 15459 | | | | | |
| 0.03 mg/ml | 15845 | | | | | |

1. A method for the care, preservation or improvement of the general state of the skin or hair; for the prophylaxis and/or prevention of human skin or human hair ageing processes; for the prophylaxis, prevention and/or reduction of premature skin ageing, of dry skin, skin unevenness, wrinkles, fine lines, rough skin or skin with large pores, and/or pigment defects or keratosis actinica; for the prophylaxis and/or treatment of diseases associated with skin ageing; for the treatment and prophylaxis of diseases associated with skin metabolism; for promoting wound healing; for the prophylaxis and/or prevention of human skin or human hair ageing processes which are associated with glycosylation of proteins; for the prophylaxis and/or treatment of diseases in the region of the human skin or human hair which are associated with glycosylation of proteins; for the prophylaxis and/or prevention of changes in the properties of the cells or extracellular matrix in the region of the skin or hair which are induced by lycosylation of proteins of the extracellular matrix; for the prophylaxis and/or treatment of skin diseases which are associated with defective keratinisation relating to differentiation and cell proliferation; for the treatment of acne vulgaris, acne comedonica, polymoiplhic acne, acne rosaceae, nodular acne, acne conylobata, age-related acne, acne occurring as a side effect, acne solaris, medicament-related acne or acne professionalis; for the treatment of defects of keratinisation, ichthyosis, ichthyosiform states, Darrier's disease, keratosis palmoplantaris, leukoplasia, leukoplasiform states, skin or mucosal (buccal) eczema (lichen); for the treatment of skin diseases which are associated with defective keratinisation and have an inflammatory and/or immunoallergic component; for the treatment of psoriasis relating to the skill, mucous membranes or finger- or toenails, or psoriatic rheumatism or skin atopy, eczema or respiratory atopy, or hypertrophy of the gums; for the prophylaxis and/or treatment of benign or malignant excrescence of the dermis or epidermis, which may be of viral origin, verruca vulgaris, verruca plana, epidermodysplasia verruciformis, oral papillomatosis, papillomatosis florida, or of excrescence which may be caused by UV radiation, basal cell epithelioma or spinal cell epithelioma, comprising administering onto the skin or hair a composition comprising an aqueous or hydroalcoholic extract of bauhinia.

2. A method according to claim 1, wherein said method is for the prophylaxis and/or prevention of human skin or human hair ageing processes; for the prophylaxis, prevention and/or reduction of premature skin ageing, of dry skin, skin unevenness, wrinkles, fine lines, rough skin or skin with large pores, and/or pigment defects or keratosis actinica.

3. A method according to claim 1, wherein said method is for the prophylaxis and/or treatment of diseases associated with skin ageing.

4. A method according to claim 1, wherein said method is for the treatment and prophylaxis of diseases associated with skin metabolism.

5. A method according to claim 1, wherein said method is for promoting wound healing.

6. A method according to claim 1, wherein said method is for the prophylaxis and/or prevention of human skin or human hair ageing processes which are associated with glycosylation of proteins.

7. A method according to claim 1, wherein said method is for the prophylaxis and/or treatment of diseases in the region of the human skin or human hair which are associated with glycosylation of proteins.

8. A method according to claim 1, wherein said method is for the prophylaxis and/or prevention of changes in the properties of the cells or extracellular matrix in the region of the skin or hair which are induced by glycosylation of proteins of the extracellular matrix.

9. A method according to claim 1, wherein said method is for the prophylaxis and/or treatment of skin diseases which are associated with defective keratinisation relating to differentiation and cell proliferation; for the treatment of acne vulgaris, acne comedonica, polymorphic acne, acne rosaceae, nodular acne, acne conglobata, age-related acne, acne occurring as a side effect, acne solaris, medicament-related acne or acne professionalis; for the treatment of defects of keratinisation, ichthyosis, ichthyosiform states, Darrier's disease, keratosis palmoplantaris, leukoplasia, leukoplasiform states, skin or mucosal (buccal) eczema (lichen); for the treatment of skin diseases which are associated with defective keratinisation and have an inflammatory and/or immunoallergic component; for the treatment of psoriasis relating to the skin, mucous membranes or finger- or toenails, or psoriatic rheumatism or skin atopy, eczema, or respiratory atopy, or hypertrophy of the gums.

10. A method according to claim 1, wherein said method is for the prophylaxis and/or treatment of benign or malignant excrescence of the dermis or epidermis, which may be of viral origin, verruca vulgaris, verruca plana, epidermodysplasia verruciformis, oral papillomatosis, papillomatosis florida, or of excrescence which may be caused by UV radiation, basal cell epithelioma or spinal cell epithelioma.

11. A method according to claim 1, wherein the composition comprises an aqueous or hlydroalcolholic extract of a bauhinia species elected from which is Bauhinia candicans, Bauhinia championii, Bauhinia fortificata (=Bauhinia forficata), Bauhinia manca, Bauhinia purpurea, Bauhinia racemosa, Bauhinia reticulata, Bauhinia tomentosa, Bauhinia variegata, Bauhinia cheilantha, Bauhinia guanensis, Bauhinia refusa, Bauhinia glauca, Bauhinia pauletia, Bauhinia ungulato, Bauhinia macrostachya or Bauhinia splendens.

12. A method according to claim 1, wherein the composition comprises an aqueous or hlydroalcolholic extract of bauhinia in an amount of from 0.001 to 20% by weight.

13. A method according to claim 1, wherein the composition comprises an aqueous or hydroalcoholic extract of bauhinia and at least one further skin-care ingredient and at least one carrier which is suitable for topical applications.

14. A method according to claim 1, wherein the composition comprises at least one further skin-care ingredient which is an antioxidant and/or vitamin, vitamin C or a derivative thereof, DL-α-tocopherol, tocopherol E acetate, nicotinic acid, pantothenic acid, biotin, a flavonoid, glutathione, coenzyme Q10, protein-bound zinc or selenium or α-lipoic acid.

15. A method according to claim 13, wherein at least one further skin-care ingredient is a pyrimidinecarboxylic acid, aryl oxime, and or ectoine.

16. A method according to claim 1, wherein the composition comprises at least one further active ingredient which is tiliroside, aspirin, lysine, arginine, carnosine, ethanolamine, aminoguanidine, pyridoxamine, thiamine pyrophosphate, kinetin, vitamin C or vitamin E.

17. A method according to claim 1, wherein the composition comprises one or more UV filters which are 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate or 2-phenylbenlzimidazole-5-sulfonic acid or potassium, sodium or a triethanolamine salt thereof, or an inorganic filter or encapsulated filter.

18. A method according to claim 1, wherein the hydroalcoholic bauhinia extract is obtained by
a) extraction of parts of bauhinia with the aid of a mixture of polar solvents and
b) subsequent discarding of the solvent.

19. A method according to claim 18, wherein the mixture of polar solvents includes water as one solvent and at least one further solvent that is methanol, ethanol, 1-propanol, 2-propanol, acetonitrile, acetone or ethyl acetate.

20. A method according to claim 1 wherein the aqueous bauhinia extract is obtained by aqueous extraction of parts of bauhinia.

21. A process for the preparation of a cosmetic or dermatological composition, comprising mixing together an aqueous or hydroalcoholic extract of bauhinia with a cosmetically or dermatologically suitable carrier.

22. A composition for preserving or improving the general state of the skin or hair, for the prophylaxis and/or prevention of human skin or human hair ageing processes, for the prophylaxis, prevention and/or reduction of premature skin ageing, of dry skin, skin unevenness, wrinkles, fine lines, rough skin or skin with large pores, and/or pigment defects or keratosis actinica, for the treatment of diseases associated with skin ageing, for the treatment of diseases associated with skin metabolism or for promoting wound healing,
comprising an aqueous or hydroalcoholic extract of bauhinia and at least one further skin-care care ingredient and at least one carrier which is suitable for topical applications.

23. A composition according to claim 22, wherein the at least one further skin-care ingredient is an antioxidant and/or vitamins vitamin C or a derivative thereof, DL-α-tocopherol, tocopherol E acetate, nicotinic acid, pantothenic acid, biotin, a flavonoid glutathione, coenzyme Q10, protein-bound zinc or selenium or α-lipoic acid.

24. A composition according to claim 22, wherein the at least one further skin-care ingredient is a pyrimidinecarboxylic acid aryl oxime, and/or ectoine.

25. A composition according to claim 22, wherein the at least one further active ingredient is tiliroside, aspirin, lysine, arginine, carnosine, ethanolamine, aminoguanidine, pyridoxamine, thiamine pyrophosphate, kinetin, vitamin C or vitamin E.

26. A composition according to claim 22, further comprising one or more UV filters which is 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate or 2-phenylbenzimidazole-5-sulfonic acid or a potassium, sodium or triethanolamine salt thereof, or an inorganic filter or encapsulated filter.

27. A method according to claim 1, wherein the composition comprises an aqueous or hydroalcoholic extract of bauhinia in an amount of from 0.01 to 10% by weight.

28. A method according to claim 19, wherein the mixture comprises 10-90% (vol.) of water.

29. A method according to claim 19, wherein the mixture comprises 30-70% (vol.) of water.
